(19) United States
(12) Patent Application Publication
(10) Pub. No.: US 2010/0016811 A1
(43) Pub. Date: Jan. 21, 2010

(54) NEEDLE TIP

(76) Inventor: Trevor Smith, Shepparton (AU)

Correspondence Address: SUGHRUE MION, PLLC, 2100 PENNSYLVANIA AVENUE, N.W., SUITE 800, WASHINGTON, DC, 20037, US

(21) Appl. No.: 12/520,322

(22) PCT Filed: Dec. 20, 2007
(86) PCT No.: PCT/AU07/01949

§ 371 Date: Jun. 19, 2009

(30) Foreign Application Priority Data
Dec. 21, 2006 (AU) 2006907103

(51) Int. Cl. A61M 5/32

(52) U.S. Cl. 604/273

(57) ABSTRACT

The present invention provides a needle tip for needles of cannulae for penetrating materials and transferring fluids. The needle tip incorporates at least one conduit for transferring the fluids, at least one blade portion and at least one aperture in fluid communication with the conduit, an aperture being disposed generally opposite a blade portion. The at least one blade portion preferably comprises of concave surfaces joining in a cutting surface. A needle tip according to the invention may have more than one conduit, more than one blade portion, and more than one aperture. Preferably, the needle tip comprises of a tear-drop shape in cross section. The needle tip is simple to make and can be advantageously used in single-use applications. Preferably the needle tip is comprised of polymeric material. Preferably the needle tip is manufactured by fluid-assisted moulding.

### FIELD OF THE INVENTION

The present invention relates to tips for needles or cannulae, in particular, tips for hypodermic needles or cannulae, and more particularly, single-use polymeric hypodermic needles or cannulae.

### BACKGROUND

The construction of needles or cannulae for transferring fluids, including substances such as gases, liquids, solutions, colloidal suspensions and the like, requires that the needles can penetrate material, such as tissue, easily to facilitate transfer of a fluidic substance but minimise damage to the material, including injury to tissue, during penetration and withdrawal of the needle. In many applications, needles are used only once, some to avoid cross-contamination between subjects and the potential for unanticipated health consequences. These performance demands for effective use of needles require the needle to be strong enough to effect penetration, but have dimensions that allow minimal damage in operation. The desire for a single use for each needle requires that needles be simple and cheap to manufacture.

The cutting edges on penetrating or cutting instruments such as needles, knives, spears and the like characteristically incorporate cutting surfaces with angles that result from a design that is a compromise between acuteness and strength, that is, the angles cannot be made too acute so as to lower penetration force that the implement is structurally weak and becomes too flimsy to penetrate target material. Developing the sharpest needle tip profile is inextricably linked with the tip as a mechanical structure.

It is known in the art to minimise manufacturing expense by making single-use needles that comprise drawn stainless steel tubing fashioned to a point by grinding a number of facets onto the end of the tube. Such needles are simple to make but require penetration forces that can cause material damage or undue tissue injury because the ‘heel’ of the needle must cut its way through target material, which results in ‘coring’ from a slug of material being forced into the conduit as the needle penetrates the material.

‘Atraumatic needles’ have been designed to overcome the coring problem. These needles do not core material in penetrating and require less penetrative force because they incorporate side ports for transfer of substances. However, such atraumatic needles are expensive to manufacture. The expense of such needles restricts their use to special applications. What is needed is a needle that is simple and cheap to manufacture while being strong enough to penetrate material such as tissue to a desired depth for substance transfer with minimal damage to the material while penetrating the tissue.

### BRIEF DESCRIPTION OF THE FIGURES

FIG. 1 shows a perspective view of needle tip having a single blade.

FIG. 2 shows a side view of a needle tip having a single blade.

FIG. 3 shows an end view of a needle tip having a single blade from the point end with the three cross sections taken from FIG. 2,

FIG. 4a shows a side view of a needle tip having a single blade.

FIG. 4b shows a side view, rotated about 45 degrees of the needle tip shown in FIG. 4a.

FIG. 4c shows a side view, rotated about 90 degrees of the needle tip shown in FIG. 4a.

FIG. 4d shows a side view of the needle tip in FIG. 4a rotated by about 180 degrees.

FIG. 5 shows a perspective view of a needle with two blades.

FIG. 6 shows a side view of a needle tip with two blades.

FIG. 7 shows an end view of a needle tip having a double blade from the point end with the four cross sections taken from FIG. 6.

FIG. 8a shows a side view of a needle tip having a double blade.

FIG. 8b shows a side view, rotated about 45 degrees of the needle tip shown in FIG. 8a.

FIG. 8c shows a side view, rotated about 90 degrees of the needle tip shown in FIG. 8a.

### SUMMARY OF THE INVENTION

It was shown by Stevens et al in U.S. Pat. No. 5,620,639, hereinafter referred to as the '639 patent, that it is possible to injection mould a polymeric needle tip integral to a conduit in a simple process. The process described in the '639 patent established the potential for developing an atraumatic needle shape at little cost for general purpose single-use hypodermic needles. The method of the '639 patent is a suitable alternative for manufacturing embodiments of the present invention.

It is an object of the invention to provide a tip for needles and the like which is relatively atraumatic in use. It is a further object of the invention to provide a needle tip that is simple to manufacture. It is a further object of the invention to provide a needle tip that is cheap to manufacture so that it can be used only once. Other objects will become apparent on reading the description of the invention described herein.

In one aspect, the invention provides a needle for penetrating material and transferring fluidic substances, the needle comprising at least one conduit portion, a tip portion comprising at least one blade portion; and at least one aperture in fluid communication with the conduit portion, said aperture disposed generally opposite the blade portion. Preferably a blade portion is comprised of two surfaces disposed at an acute angle. Preferably the two surfaces are disposed at an acute angle ranging from about 10 degrees to about 20 degrees. Preferably, a blade portion of a needle comprises of concave surfaces. Preferably, the tip portion comprises of a substantially tear-drop shape in cross section. Preferably, the needle includes a sharp point at one end. Preferably, the needle is comprised of a polymeric material. More preferably, the polymeric material is suitable for injection moulding or compression moulding.

### DETAILED DESCRIPTION OF THE INVENTION AND MOST PREFERRED EMBODIMENTS

The present invention provides a needle comprising of a tip portion incorporating at least one blade, at least one aperture for substance transfer, conduit portion in fluid communication with the aperture, the needle being relatively solid and strong while providing a sharp point to effect penetration. The needle is suitable for use in many types of applications involving the transfer of fluids. Such fluids may be liquids or gases, including solutions, colloids, and suspensions of particulate matter in fluids or gases. The advantageous properties of the needle in construction and composition that make it strong but relatively atraumatic in use, causing less damage to the material it penetrates, as well as being cheap and easy to manufacture, make it suitable for transferring fluids to and from bodies, or to and from containers, as well as many other applications that will become evident to a broad range of users.

The present invention incorporates the surprising observation that if a needle tip comprises of a very long cutting edge generally opposing a side delivery aperture or port, the blade angle can be made very acute without sacrificing rigidity or weakening of the tip. Structurally, such a blade acts as a reinforcing rib and is itself supported by the rest of the tip geometry. A side port in a blade potentially creates structural weakness in the material. However, if the mass of the tip is biased over to support the aperture this can be done by making the cutting angle of the long blade more acute, the result is a needle tip that is both relatively very strong and sharp. Further, if this single long blade is given a concave form, cutting angles can be made very acute without much loss in rigidity of the tip, typically 10-20 degrees of Included angle.

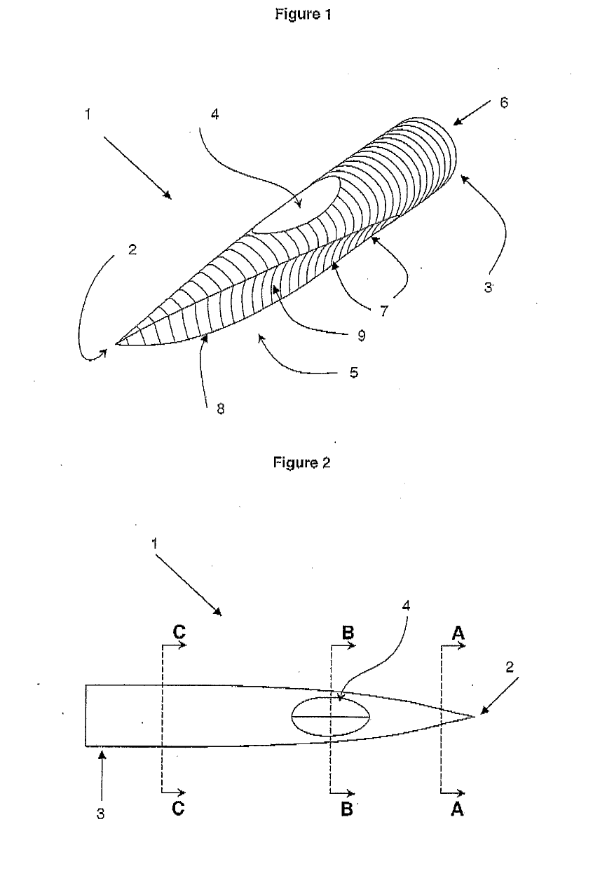

The invention is better understood, with reference to the figures. In this description to the figures illustrate embodiments of the invention. It will be understood that further embodiments within the spirit and scope of the invention are possible and that the scope of the invention is limited only by the claims appended hereto. FIG. 1 shows a perspective side view of an embodiment of a three-dimensional form of a needle tip 1, comprising of a point end 2, distal end 3, and blade portion 5. The blade portion is comprised of generally curved surfaces 9 that meet to form a generally curved cutting edge 8. Fluidic material is transferred from the distal end 3 to the port 4 through the conduit portion 6. It will be understood that the port 4 may act as an inlet or outlet for fluidic material, which may move in either direction through the conduit portion 6, depending on whether pressure or suction is applied to the conduit. It will be understood that the conduit may take other embodiments, such as being comprised of more than one passage. Multiple passages may be concentrically disposed or side-by-side. A series of parallel lines 7 are included in the drawing to conveniently define the surface of an embodiment of a needle tip 1 according to the invention.

Preferably, the blade is comprised of concave surfaces as shown in FIG. 1. However, the scope of the invention includes that the curved surfaces 9 of the blade portion 5 may be varied, including but not limited to flat or convex surfaces. The construction of the needle tip 1 advantageously results in minimal drag of blade portion 5 as the tip and cutting edge 8 open an entry into a material, including tissue, starting from an initial point of contact and incising the material up to the diameter of the conduit at the distal end 3. The needle tip incorporates a side aperture or port 4 in disposed generally in a surface opposite the blade portion 5. Preferably the needle is comprised of a polymeric material. Preferably the material is suitable for injection moulding or compression moulding of any type, for example fluid-assisted moulding, two-shot moulding, thermoplastic or thermosetting moulding.

FIG. 2 shows a representative side view of a needle tip 1 with point end 2, distal end 3 and port 4. Dotted lines at A-A, B-B, and C-C define three cross sections perpendicular to the longitudinal axis of the needle tip 1. Each of said cross sections intersects a different portion of the needle tip near the distal end C-C, the port 4 B-B, and near the point end 2 A-A.

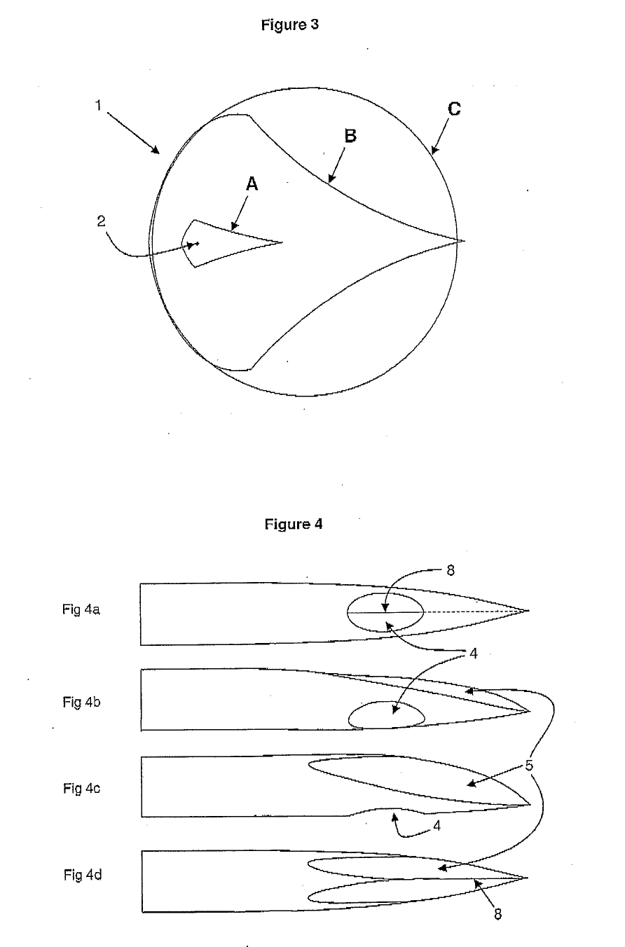

FIG. 3 shows a view from the point end 2 of the needle tip 1 perpendicular to the side view in FIG. 2. From the viewer's perspective the longitudinal axis of the needle tip projects into and behind the page. The cross sections at A-A, B-B, and C-C are represented by the contour lines A, B, and C, respectively, in FIG. 3. The contour lines A, B, and C define the perimeters of three parallel planes along the longitudinal axis of the needle tip 1, where the plane defined by contour line A is located nearest the tip point 2, the plane defined by contour line C is most distal, and the plane defined by contour line B is intermediate between the other two. The port 4 includes the plane defined by contour line B.

The relationship of the planes and surface shape of the needle tip in three dimensions can be understood by inspection of FIGS. 2 and 3. In this embodiment, the cross-sectional planes defined by the contour lines along the perimeters at A and B define generally “tear drop” shapes. Similarly all intermediate planes define generally tear drop shapes in cross section. The tear drop shape of the perimeter of the needle tip gradually smooths to the shape of a circle at contour line C in the embodiments shown in the figures included with this specification. It will be understood that the scope of the invention includes that the perimeter at C and more distal portions of the needle tip may take whatever suitable shape is required for application for the needle tip.

The tear drop shape in cross-section through and near the side port is a surprisingly efficient means of incising and opening up material such as tissue as the needle tip penetrates the material. The invention conveniently results in the major forces involved in penetrating the material being directed along the cutting edge of the blade because the sides of the blade are relieved from contacting the material being penetrated. Preferably the needle comprises of a sharp point at a distal end as illustrated herein. However, the scope of the invention includes that the distal end includes alternative shapes. From a sharp point at a distal end, the needle tip preferably forms a tear-shaped portion in cross section along the longitudinal axis of the tip, the tear-shaped portion being similar to the cross-section shown in FIG. 1 and then graduating to the round cross-section of the conduit.

The port 4 is continuous with the conduit formed by the internal surface of needle tip. The shape of the port is generally defined by the angle of the perimeter of the port and the longitudinal axis of the needle tip. Generally, the area of the port is approximately the same as the cross-sectional area of the conduit.

FIG. 4 shows in side view a needle tip at four orientations showing the positioning of the port 4 and the blade portion 5. In FIG. 4a the port can be seen relative to the cutting edge 8, shown as a dashed line. In FIG. 4b the needle tip has been rotated to a position approximately 45 degrees relative to that in FIG. 4a so that the blade portion 5 is visible. In FIG. 4c the needle tip has been rotated to a position that is approximately 90 degrees relative to that in FIG. 4a so that the port 4 is near the bottom of the needle tip and the blade portion is oriented opposite the port 4. In FIG. 4d the needle tip has been rotated to a position that is approximately 180 degrees relative to that in FIG. 4a and is the opposite side to that shown in FIG. 4a. In FIG. 4d the cutting edge 8 is in approximately the centre of the needle tip.

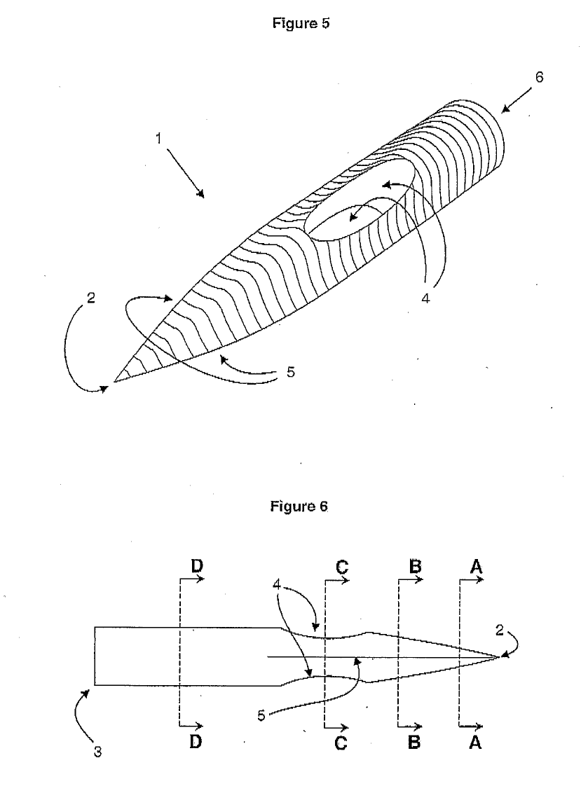

The scope of the invention is not limited to having a single port and single blade in a needle tip as shown in FIGS. 1 to 4. Other embodiments are within the scope, including those shown in FIGS. 5 to 8. The scope of the invention includes that even further embodiments that are not illustrated in this document, including a plurality of blades and a plurality of ports.

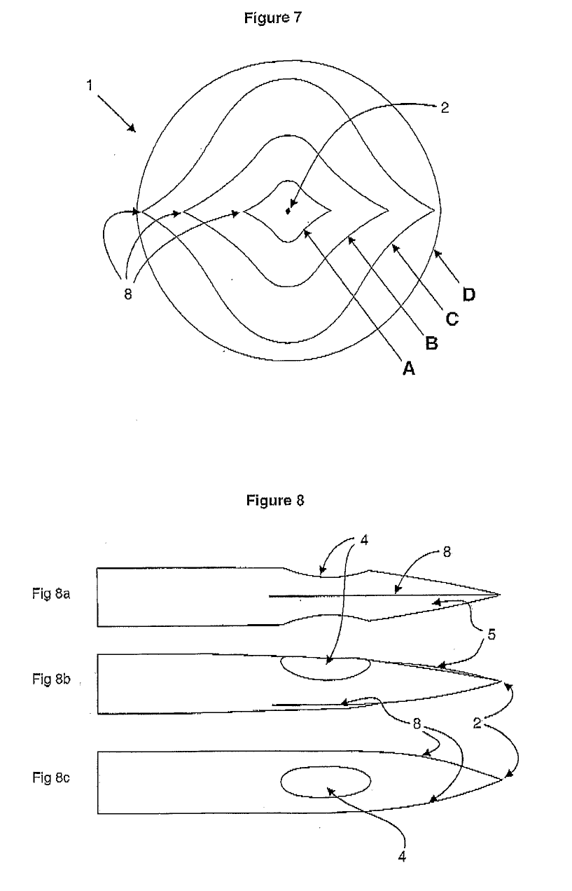

FIG. 5 shows an embodiment of the invention in perspective view as a needle tip incorporating two blade portions 5. The needle tip further incorporates two ports 4. Each port is continuous with the conduit. FIG. 6 shows in side view the double-bladed needle tip, the view being similar to that shown in FIG. 2. The dotted lines in FIG. 6 at A-A, B-B, C-C, and D-D represent cross-sections taken perpendicular to the transverse longitudinal section. The cross-section at A-A is taken near the tip point 2 and the cross-section at D-D is taken nearer the distal end 3 of the needle tip. The cross-section at C-C is taken at a plane that is perpendicular and bisects the two opposite ports 4, which have openings generally perpendicular to the blade portions 5. FIG. 7 shows a similar view to FIG. 3 of the needle tip 1 show in FIG. 5. That is, the tip point 2 is near the centre of FIG. 7 and the surface of the needle tip extends to incorporate the perimeters shown at A, B, C, and D. Each of the blade portions 5 incorporates a cutting edge 8 formed by the two surfaces of each blade portion.

FIG. 8, similar to FIG. 4, shows in side perspective view a needle tip incorporating two ports 4 between two blades 5, The surprising strength of the needle tip resulting from the blade portion 5 being positioning adjacent a port can be expanded to multiple blades and multiple ports. FIG. 8 shows in side perspective view a needle tip at three orientations showing the positioning of the ports 4 and the blade portions 5. In FIG. 8a the ports can be seen above and below relative to the cutting edge 8 on a first side. The opposite side incorporating the opposite cutting edge 8 cannot be seen in the figure. In FIG. 8b the needle tip has been rotated to a position approximately 45 degrees to that in FIG. 8a so that the blade portion 5 is visible. One cutting edge 8 can be seen near the bottom of the needle tip. In FIG. 8c the needle tip has been rotated to a position that is approximately 90 degrees to that in FIG. 8a so that the port 4 is centred in the needle tip and the blade portions 5 and cutting edges 8 are at the top and bottom of the needle tip.

1. A needle for penetrating material and transferring fluidic substances comprising:
at least one conduit portion;
a tip portion comprising at least one blade portion; and
at least one aperture in fluid communication with a conduit portion, said
at least one aperture disposed generally opposite a blade portion.

2. The needle according to claim 1, the blade portion comprising of two surfaces disposed at an acute angle.

3. The needle according to claim 2, the acute angle ranging from about 10 degrees to about 20 degrees.

4. The needle according to claim 1, the blade portion comprising of concave surfaces.

5. The needle according to claim 3, the tip portion comprising of a substantially tear-drop shape in cross section.

6. The needle according to claim 1, further including a sharp point at one end.

7. The needle according to claim 1, the needle comprised of a polymeric material.

8. The needle according to claim 6, the polymeric material being suitable for injection moulding.

9. The needle according to claim 7, the polymeric material being suitable for injection moulding or compression moulding.

10. A needle for penetrating material and transferring fluidic substances, the needle substantially as herein described with reference to the figures.

11. The needle according to claim 2, the blade portion comprising of concave surfaces.

12. The needle according to claim 3, the blade portion comprising of concave surfaces.

13. The needle according to claim 4, the tip portion comprising of a substantially tear-drop shape in cross section.

14. The needle according to claim 5, further including a sharp point at one end.

15. The needle according to claim 6, the needle comprised of a polymeric material.

16. The needle according to claim 8, the polymeric material being suitable for injection moulding or compression moulding.
